(19) United States
(12) Patent Application Publication
(10) Pub. No.: US 2021/0259302 A1
(43) Pub. Date: Aug. 26, 2021

(54) CIGARETTE ADHESIVE INCLUDING MEDICINAL OR RECREATIONAL SUBSTANCES

(71) Applicant: Top Shelf Tobacco Services Limited, Fleet (GB)

(72) Inventors: Kelly Ritter, III, Wilmington, NC (US); Andrew Neil Blay, Fleet (GB)

(21) Appl. No.: 16/859,761

(22) Filed: Apr. 27, 2020

Related U.S. Application Data
(60) Provisional application No. 62/980,746, filed on Feb. 24, 2020.

(51) Int. Cl. A24D 1/02; A24D 1/18; A24D 1/22; A61K 31/05; C09J 11/06

(52) U.S. Cl. CPC A24D 1/025; A24D 1/18; C09J 11/06; A61K 31/05; A24D 1/22

(57) ABSTRACT

A smoking article, such as a cigarette or cigar, having an outer layer and a combustible substance disposed within the outer layer. An adhesive is applied to at least a portion of the outer layer around the combustible substance to create a rolled smoking article. The adhesive includes a predetermined dosage of a cannabinoid, such as, but not limited to, phytocannabinoid thetrahydrocannabinol, cannabidiol, and cannabigerol.

### PRIORITY

This application claims benefit of U.S. Provisional Application No. 62/980,746, titled “CIGARETTE ADHESIVE INCLUDING CANNABINOIDS,” filed on Feb. 24, 2020, which is incorporated herein by reference in its entirety.

### BACKGROUND

Cigarettes, cigars, or otherwise are popular smoking articles that can include combustible hemp or marijuana, which can be used to consume medical or recreational cannabis. However, each cannabis plant has its own individual Phytocannabinoid properties based on seed variety, soil type, weather conditions, times of harvesting and other agronomical practices. The phytocannabinoid properties can even vary based on the specific position on the plant a flower cluster, Bud or leaf is taken from. This results in combustible hemp or marijuana having variable levels of cannabinoids which can make it difficult to regulate how much cannabinoid a user is inhaling when smoking combustible cannabis. Moreover, synthetic cannabinoids are available and can help deliver a more regulated dose.

Embodiments of the disclosure address these and other deficiencies of the prior art.

### BRIEF DESCRIPTION OF THE DRAWINGS

Aspects, features and advantages of embodiments of the present disclosure will become apparent from the following description of embodiments in reference to the appended drawings in which:

FIG. 1 illustrates an example of an unrolled cigarette or cigar.

FIG. 2 illustrates an example of a rolled cigarette or cigar.

### DESCRIPTION

Embodiments of the disclosure include medicinal or recreational substances, such as cannabinoids either naturally extracted from Cannabis plants or synthetically produced or opiates, included in the paper adhesive to accurately regulate how much medicinal or recreational substance is included with each combustible. By including a pre-defined dosage of medicinal or recreational substance in the adhesive, the amount of medicinal or recreational substance inhaled or consumed can be regulated when the unit is combusted.

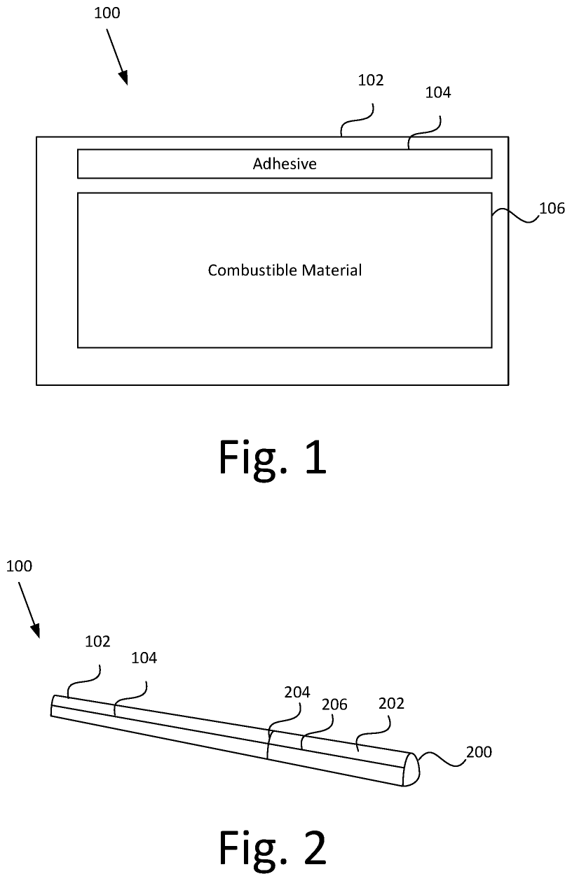

FIG. 1 is an example of an unrolled smoking article, such as a cigarette or cigar 100, for ease of illustration. As will be understood by one skilled in the art, a cigarette or cigar can have a number of other components not illustrated in FIG. 1 for ease of discussion. Some of these components are discussed below and illustrated in FIG. 2. Further, as discussed in detail below, embodiments of the disclosure are not limited to rolled cigarettes or cigars, but may also include other smoking articles, such as but not limited to, cigarette tubes, pre-rolls and/or cones, cigarette papers, and blunt wraps.

The unrolled cigarette or cigar 100 includes cigarette or homogenized paper 102 or outer wrapper that can be rolled to in a cylindrical rod shape. An adhesive 104 mixed with one or more medicinal or recreational substances at a predetermined dosage is applied to the edge of the paper 102. The one or more medicinal or recreational substances may be, for example, a cannabinoid or an opiate in a predetermined dosage. The substances have a known, pre-determined potency or dosage prior to being added or mixed with the adhesive 104. Examples of cannabinoids may include, but are not limited to, phytocannabinoid thetrahydrocannabinol (THC), cannabidiol (CBD), and/or cannabigerol (CBG). However, any known or unknown cannabinoid may be added to the adhesive.

Prior to being rolled, a combustible substance 106 is placed on the paper 102. However, if a pre-rolled cigarette or tube is used, the combustible substance 106 may be included after the cigarette is rolled or formed. The combustible substance 106 may be placed on the paper 102 before or after the adhesive 104. The combustible substance 106 can be any combustible substance 106 that has a controllable burn in conjunction with the paper. The combustible substance 102, however, does not necessarily include any cannabinoids.

When the paper 102 is rolled in the cylindrical rod or cone shape, the adhesive 104 with the medicinal or recreational substances adheres the sides of the cigarette paper 102 together to form the cigarette or cigar 100, as illustrated in FIG. 2. FIG. 2 illustrates the cigarette or cigar 100 rolled in a cylindrical rod shape. FIG. 2 illustrates some of the additional components that may be included in the cigarette or cigar. For example, the combustible may also include a filter 200 wrapped in tipping papers 202. The filter 200 may be adhered to the paper 102 by a filter adhesive 204. The cigarette or cigar 100 may also include a plug wrap 206. Within the rolled paper 102 is also the combustible material 106. The adhesive 104 containing the medicinal or recreational substance may be used only on the overlap or sideseam edge of the smoking column. In general machine made cigarette or cigar manufacturing the filter 200 is a separate component that is added to the smoking column during the making and packing process. The filter paper 206 overlaps on the outside of the smoking column when filter and the smoking column sections are put together. As such there is no reason to use adhesive containing the medicinal or recreational substance outside the smoking column as this part of the cigarette or cigar is not combusted. However, in some embodiments, the adhesive 104 may be used on the filter for ease of manufacturing.

Any type of adhesive may be used and there are many different known methods for applying the adhesive 104 to the paper 102. Any method for applying the adhesive to the paper may be used, so long as the one or more medicinal or recreational substances are added to the adhesive 104 prior to the application on the paper 102.

If cannabis is used, the cannabinoids may be extracted from the cannabis plant using any known method or may be synthetically produced. The cannabinoids may be distillation in isolate, distillate, full spectrum, or broad spectrum.

Prior to combining the one or more medicinal or recreational substances to the adhesive, the medicinal or recreational substances can be tested and it can be determined the amount of one or more medicinal or recreational substances needed to be included in the adhesive to achieve a particular medicinal or recreational substances dosage. This can allow for the delivery effects of the medicinal or recreational substances to be regulated and controlled on a wide and easily repeatable scale.

For example, medical marijuana users may desire or require a particular dosage of a cannabinoid as directed by a medical professional for medicinal purposes. Recreational users may desire a particular dosage of a cannabinoid, and using conventional cannabis cigarettes can result in a person receiving more or less cannabinoid than desired. Further, recreational use of marijuana may be legal in some geographies, but only at or below particular strengths or dosages.

Embodiments of the disclosure allow for an exact or near exact dosage to be known in the cigarette or cigar. The controlled burn of the paper 102 when ignited regulates the desired medicinal or recreational substances delivery through inhalation and absorption into the blood stream of a user. This can ensure that a particular dosage of medicinal or recreational substances is correct for medical use or to ensure that the recreational use of the cannabinoid is within the legal requirements for particular geographies.

Since the one or more medicinal or recreational substances are combined with or included with the adhesive 104, the adhesive 104 may not be applied end-to-end to the combustible paper 102, but may only be applied to the portion of the paper 102 which is burned. If needed, additional adhesive without the one or more medicinal or recreational substances may be added to the end of the paper 102 which is held by a user and does not burn.

There are a number of different cigarette or cigar types that can include embodiments of the disclosure. For example, embodiments of the disclosure can be included in cigarette tubes, pre-rolls and/or cones, cigarette papers, and blunt wraps.

Cigarette tubes are empty cigarette tubes that can be adhered with the adhesive discussed above including the medicinal or recreational substances. A small combustible material insertion machine or device can put the combustible material inside the tube. Each cigarette tube is different depending on length various sizes and also different strength filters etc.

Pre-rolls and/or cones are similar to cigarette tubes. The pre-rolls can be bought pre-rolled in different lengths. The pre-rolls would be rolled with an adhesive containing a specified amount of medicinal or recreational substances. Pre-rolls can be pre rolled in different lengths and with different sized filters and the combustible material can be packed inside by the consumer.

As discussed above, cigarette papers include an adhesive with a predetermined amount of medicinal or recreational substances. A consumer can wet the adhesive to construct the cigarette and the medicinal or recreational substances would be consumed by the combustion of the cigarette.

This written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention. Accordingly, the invention should not be limited except as by the appended claims.

We claim:

1. A cigarette, comprising:
a combustible material;
an outer wrapper surrounding the combustible material; and
an adhesive applied to at least a portion of the outer wrapper, the adhesive including a predetermined dosage of a cannabinoid or opiate.

2. The cigarette of claim 1, wherein the cannabinoid is phytocannabinoid thetrahydrocannabinol.

3. The cigarette of claim 1, wherein the cannabinoid is cannabidiol.

4. The cigarette of claim 1, wherein the cannabinoid includes phytocannabinoid thetrahydrocannabinol and cannabidiol.

5. The cigarette of claim 1, wherein the adhesive is a first adhesive, the cigarette further comprising a second adhesive applied to a portion of the outer wrapper that does not include the first adhesive.

6. The cigarette of claim 1, wherein the adhesive is applied along an edge of the outer wrapper.

7. The cigarette of claim 1, wherein the cannabinoid is synthetically produced.

8. The cigarette of claim 1, wherein the cannabinoid is cannabigerol.

9. A smoking article, comprising:
an outer layer;
a combustible substance disposed within the outer layer; and
an adhesive including a predetermined dosage of a medicanal or recreational substance applied to at least a portion of the outer layer around the combustible substance.

10. The smoking article of claim 9, wherein the medicinal or recreational substance is phytocannabinoid thetrahydrocannabinol.

11. The smoking article of claim 9, wherein the medicinal or recreational substance is cannabidiol.

12. The smoking article of claim 1, wherein the adhesive includes a predetermined dosage of multiple medicinal or recreational substances.

13. The smoking article of claim 9, wherein the adhesive is a first adhesive, the smoking article further comprising a second adhesive applied to a portion of the outer layer that does not include the first adhesive.

14. The smoking article of claim 9, wherein the adhesive is applied along an edge of the outer layer.

15. The smoking article of claim 9, wherein the medicinal or recreational substance is a synthetically produced cannabinoid.

16. The smoking article of claim 9, wherein the medicinal or recreational substance is cannabigerol.

17. A method for including a medicinal or recreational substance in a smoking article, the method comprising:
applying an adhesive to an edge of an outer layer of the smoking article;
applying a predetermined dosage of a medicinal or recreational substance to the adhesive; and
adhering the edge of the outer layer having the adhesive with an edge of the outer layer not having an adhesive to form the smoking article.

18. The method of claim 17, wherein the medicinal or recreational substance includes one more of phytocannabinoid thetrahydrocannabinol, cannabidiol, and cannabigerol.

19. The method of claim 17, wherein the adhesive includes a predetermined dosage of multiple medicinal or recreational substance.

20. The method of claim 17, wherein the medicinal or recreational substance is synthetically produced.
